(19) United States
(12) Patent Application Publication
(10) Pub. No.: US 2025/0174360 A1
(43) Pub. Date: May 29, 2025

(54) CHEMICAL INFORMATION IN HEALTH INDEX

(71) Applicant: Cardiac Pacemakers, Inc., St. Paul, MN (US)

(72) Inventors: Viktoria A. Averina, Shoreview, MN (US); Yingbo Li, Woodbury, MN (US); Michael J. Kane, St Paul, MN (US); Pramodsingh Hirasingh Thakur, Woodbury, MN (US)

(21) Appl. No.: 18/958,518

(22) Filed: Nov. 25, 2024

Related U.S. Application Data
(60) Provisional application No. 63/604,098, filed on Nov. 29, 2023.

(51) Int. Cl. G16H 50/30; A61B 5/00; G16H 10/60

(52) U.S. Cl. CPC G16H 50/30; A61B 5/686; A61B 5/742; A61B 5/746; G16H 10/60

(57) ABSTRACT

Systems and methods are disclosed to determine or adjust a health index for a patient, including receiving physiologic information of the patient, receiving chemical information of the patient, determining a composite health index for the patient as a weighted function of one or more physiologic features of the received physiologic information and one or more chemical features of the received chemical information, and generating a composite health index alert as a function of the determined composite health index and an alert threshold.

### CLAIM OF PRIORITY

This application claims the benefit of U.S. Provisional Application No. 63/604,098, filed on Nov. 29, 2023, which is hereby incorporated by reference in its entirety.

### TECHNICAL FIELD

This document relates generally to medical devices and more particularly to determining a health index including chemical information.

### BACKGROUND

Ambulatory medical devices (AMDs), including implantable, subcutaneous, wearable, or one or more other medical devices, etc., can monitor, detect, or treat various conditions, including heart failure (HF), atrial fibrillation (AF), etc. Ambulatory medical devices can include sensors to sense physiological information from a patient and one or more circuits to detect one or more physiologic events using the sensed physiological information or transmit sensed physiologic information or detected physiologic events to one or more remote devices. Frequent patient monitoring can provide early detection of worsening patient condition, including worsening heart failure or atrial fibrillation.

Accurate identification of patients or groups of patients at an elevated risk of future adverse events may control mode or feature selection or resource management of one or more ambulatory medical devices, control notifications or messages in connected systems to various users associated with a specific patient or group of patients, organize or schedule physician or patient contact or treatment, or prevent or reduce patient hospitalization. Correctly identifying and safely managing patient risk of worsening condition may avoid unnecessary medical interventions, extend the usable life of ambulatory medical devices, and reduce healthcare costs.

### SUMMARY

Systems and methods are disclosed to determine or adjust a health index for a patient, including receiving physiologic information of the patient, receiving chemical information of the patient, determining a composite health index for the patient as a weighted function of one or more physiologic features of the received physiologic information and one or more chemical features of the received chemical information, and generating a composite health index alert as a function of the determined composite health index and an alert threshold. In certain examples, the chemical information can include one or more of potassium information or creatinine information.

An example of subject matter (e.g., a medical device system) may comprise a signal receiver circuit configured to receive physiologic information of a patient and chemical information of the patient, and an assessment circuit configured to determine a composite health index for the patient as a weighted function of one or more physiologic features of the received physiologic information and one or more chemical features of the received chemical information, and generate a composite health index alert as a function of the determined composite health index and an alert threshold.

In an example, the assessment circuit is configured to determine the composite health index for the patient as a function of at least two features of the received physiologic information and one or more features of the received chemical information.

In an example, which may be combined with any one or more of the previous examples, the one or more chemical features comprise one or more indications of relative high or low values of the received chemical information with respect to one or more chemical feature thresholds.

In an example, which may be combined with any one or more of the previous examples, the assessment circuit is configured to determine the one or more chemical feature thresholds as a function of the received chemical information over a first time period and determine the indication of relative high or low values of the received chemical information using a comparison of a value of the received chemical information to the determined one or more chemical feature thresholds.

In an example, which may be combined with any one or more of the previous examples, the assessment circuit is configured to determine a weight of the one or more chemical features as a function of a value of the one or more chemical features.

In an example, which may be combined with any one or more of the previous examples, the assessment circuit is configured to determine a value of the composite health index that exceeds a value of the alert threshold for a first range of values of the one or more chemical features, independent of the one or more physiologic features.

In an example, which may be combined with any one or more of the previous examples, the assessment circuit is configured to determine the alert threshold as a function of the received chemical information.

In an example, which may be combined with any one or more of the previous examples, the assessment circuit is configured to determine the alert threshold to increase a sensitivity of the composite health index alert for a second range of values of the one or more chemical features.

In an example, which may be combined with any one or more of the previous examples, the assessment circuit is configured to determine the one or more physiologic features or the one or more chemical features of the weighted function of the composite health index as a function of one or more values of the one or more chemical features.

In an example, which may be combined with any one or more of the previous examples, the assessment circuit is configured to increase a number of features of the one or more physiologic features or the one or more chemical features of the weighted function for a third range of values of the one or more chemical features.

In an example, which may be combined with any one or more of the previous examples, the composite health index includes a composite heart failure index and the composite health index alert threshold includes a composite heart failure alert threshold, at least one of the one or more chemical features or the chemical information includes at least one of potassium information or creatinine information of the patient, and the assessment circuit is configured to provide an output of the generated composite health index alert to a user interface for display to a user or to a control circuit to control or adjust a process or function of the medical device system.

In an example, which may be combined with any one or more of the previous examples, a method includes receiving, using a signal receiver circuit, physiologic information of a patient and chemical information of the patient, determining, using an assessment circuit, a composite health index for the patient as a weighted function of one or more physiologic features of the received physiologic information and one or more chemical features of the received chemical information, and generating, using the assessment circuit, a composite health index alert as a function of the determined composite health index and an alert threshold.

In an example, which may be combined with any one or more of the previous examples, the one or more chemical features comprise one or more indications of relative high or low values of the received chemical information with respect to one or more chemical feature thresholds, the method comprises determining, using the assessment circuit the one or more chemical feature thresholds as a function of the received chemical information over a first time period, and determining the indication of relative high or low values of the received chemical information by comparing a value of the received chemical information to the determined one or more chemical feature thresholds.

In an example, which may be combined with any one or more of the previous examples, the method includes determining, using the assessment circuit, a weight of the one or more chemical features as a function of a value of the one or more chemical features, and determining, using the assessment circuit, a value of the composite health index that exceeds a value of the alert threshold for a first range of values of the one or more chemical features, independent of the one or more physiologic features.

In an example, which may be combined with any one or more of the previous examples, the method includes determining, using the assessment circuit, the alert threshold as a function of the received chemical information including determining the alert threshold to increase a sensitivity of the composite health index alert for a second range of values of the one or more chemical features.

In an example, which may be combined with any one or more of the previous examples, a medical device system can include a signal receiver circuit configured to receive physiologic information of a patient, and chemical information of the patient, and an assessment circuit configured to determine a composite health index for the patient as a weighted function of one or more physiologic features of the received physiologic information and one or more chemical features of the received chemical information and generate a composite health index alert as a function of the determined composite health index and an alert threshold.

In an example, which may be combined with any one or more of the previous examples, the assessment circuit is configured to determine the composite health index for the patient as a function of at least two features of the received physiologic information and one or more features of the received chemical information.

In an example, which may be combined with any one or more of the previous examples, the one or more chemical features comprise one or more indications of relative high or low values of the received chemical information with respect to one or more chemical feature thresholds.

In an example, which may be combined with any one or more of the previous examples, the assessment circuit is configured to determine the one or more chemical feature thresholds as a function of the received chemical information over a first time period, and determine the indication of relative high or low values of the received chemical information using a comparison of a value of the received chemical information to the determined one or more chemical feature thresholds.

In an example, which may be combined with any one or more of the previous examples, the assessment circuit is configured to determine a weight of the one or more chemical features as a function of a value of the one or more chemical features.

In an example, which may be combined with any one or more of the previous examples, the assessment circuit is configured to determine a value of the composite health index that exceeds a value of the alert threshold for a first range of values of the one or more chemical features, independent of the one or more physiologic features.

In an example, which may be combined with any one or more of the previous examples, the assessment circuit is configured to determine the alert threshold as a function of the received chemical information.

In an example, which may be combined with any one or more of the previous examples, the assessment circuit is configured to determine the alert threshold to increase a sensitivity of the composite health index alert for a second range of values of the one or more chemical features.

In an example, which may be combined with any one or more of the previous examples, the assessment circuit is configured to determine the one or more physiologic features or the one or more chemical features of the weighted function of the composite health index as a function of one or more values of the one or more chemical features.

In an example, which may be combined with any one or more of the previous examples, the assessment circuit is configured to increase a number of features of the one or more physiologic features or the one or more chemical features of the weighted function for a third range of values of the one or more chemical features.

In an example, which may be combined with any one or more of the previous examples, the composite health index includes a composite heart failure index and the composite health index alert threshold includes a composite heart failure alert threshold, and at least one of the one or more chemical features or the chemical information includes at least one of potassium information or creatinine information of the patient.

In an example, which may be combined with any one or more of the previous examples, the assessment circuit is configured to provide an output of the generated composite health index alert to a user interface for display to a user or to a control circuit to control or adjust a process or function of the medical device system.

In an example, which may be combined with any one or more of the previous examples, a method includes receiving, using a signal receiver circuit, physiologic information of a patient and chemical information of the patient, determining, using an assessment circuit, a composite health index for the patient as a weighted function of one or more physiologic features of the received physiologic information and one or more chemical features of the received chemical information, and generating, using the assessment circuit, a composite health index alert as a function of the determined composite health index and an alert threshold.

In an example, which may be combined with any one or more of the previous examples, determining the composite health index for the patient includes determining the composite health index for the patient as a function of at least two features of the received physiologic information and one or more features of the received chemical information.

In an example, which may be combined with any one or more of the previous examples, the one or more chemical features comprise one or more indications of relative high or low values of the received chemical information with respect to one or more chemical feature thresholds, the method comprises determining, using the assessment circuit the one or more chemical feature thresholds as a function of the received chemical information over a first time period, and the indication of relative high or low values of the received chemical information by comparing a value of the received chemical information to the determined one or more chemical feature thresholds.

In an example, which may be combined with any one or more of the previous examples, the method includes determining, using the assessment circuit, a weight of the one or more chemical features as a function of a value of the one or more chemical features.

In an example, which may be combined with any one or more of the previous examples, the method includes determining, using the assessment circuit, a value of the composite health index that exceeds a value of the alert threshold for a first range of values of the one or more chemical features, independent of the one or more physiologic features.

In an example, which may be combined with any one or more of the previous examples, the method includes determining, using the assessment circuit, the alert threshold as a function of the received chemical information.

In an example, which may be combined with any one or more of the previous examples, the method includes determining, using the assessment circuit, the alert threshold to increase a sensitivity of the composite health index alert for a second range of values of the one or more chemical features.

In an example, which may be combined with any one or more of the previous examples, the method includes determining, using the assessment circuit, the one or more physiologic features or the one or more chemical features of the weighted function of the composite health index as a function of one or more values of the one or more chemical features.

In an example, a system or apparatus may optionally combine any portion or combination of any portion of any one or more of the examples above to comprise “means for” performing any portion of any one or more of the functions or methods of the examples above, or at least one “non-transitory machine-readable medium” including instructions that, when performed by a machine, cause the machine to perform any portion of any one or more of the functions or methods of the examples above.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example representation of determining a chemical feature based on received chemical information.

FIG. 2 illustrates an example medical device system.

FIG. 3 illustrates an example patient management system.

FIG. 4 illustrates an example method for using chemical information to determine a health index.

FIG. 5 illustrates an example implantable medical device (IMD) electrically coupled to a heart.

FIG. 6 illustrates a block diagram of an example machine upon which any one or more of the techniques discussed herein may perform.

### DETAILED DESCRIPTION

Ambulatory medical devices can include or be configured to receive physiologic information from one or more sensors located within, on, or proximate to a body of a patient. Physiologic information of the patient can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient.

One or more health indexes can be determined, in certain examples, as a function of different physiologic information of the patient or various combinations thereof. Health indexes can include single-feature health indexes determined using a single feature or measure of a single type of physiologic information, or separately a composite health index determined using a combination of physiologic information, such as two or more separate features of different physiologic measures. For example, although respiratory rate and tidal volume are both respiratory information, they are separate features of respiratory information, such that a composite health index can be determined using respiratory rate and tidal volume. In contrast, a single-feature health index can be determined using respiratory information, such as using a trend or measure of tidal volume alone.

In certain examples, a health index can be a device-based index, such as determined using physiologic information detected from the patient without input of clinical information about the patient separate from that detected or sensed from the device, such as clinician diagnosis or determination of risk, patient history, patient age, comorbidities, prior hospitalization, type of implanted device, etc. In other examples, the health index can be a combination of a device-based and clinical-based mortality risk index, including or taking into account clinical information about the patient, such as clinician diagnosis or determination of risk, patient history, patient age, comorbidities, prior hospitalization, type of implanted device, etc. In an example, separate determinations can be made for different combinations of clinical information.

One example of a composite health index is a HeartLogic™ index, a HeartLogic™ in-alert time, or one or more other composite measurements or measures thereof. The HeartLogic™ index is a composite measurement of electrical and mechanical physiologic information of a patient from multiple ambulatory sensors, including S1 and S3 heart sounds, thoracic impedance, activity information, respiration information, and nighttime heart rate (nHR), and can be indicative of a heart failure status, a risk of heart failure event, or a worsening of the heart failure status or risk of heart failure event in the patient over time. The HeartLogic™ in-alert time is a measure of time that the HeartLogic™ index is above an alert threshold.

In certain examples, the HeartLogic™ index can be determined using different combinations or weightings of electrical and mechanical physiologic information, including one or more of S1 and S3 heart sounds, thoracic impedance, activity information, rapid shallow breathing index (RSBI), respiratory rate, and nighttime heart rate (nHR). In certain examples, the different combinations or weightings of the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.).

For example, if the risk stratifier is low, or below a first threshold, the HeartLogic™ index can be determined using a first combination of physiologic information. If the risk stratifier is high, or above a second threshold, the HeartLogic™ index can be determined using the first combination of physiologic information and a second combination of physiologic information, including additional information than included in the first combination. If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

Implantable and ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc.

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a ratio that measures the respiratory frequency divided by the relative tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical physiologic information, but different than cardiac electrical information. In other examples, respiration information of the patient can be determined using changes in activity or acceleration information and accordingly can be considered mechanical physiologic information.

Physiologic values or features, as described herein, can include one or more different measures of rate, amplitude, energy, etc., of different physiologic information over one or more time periods, such as representative daily values, etc. For example, heart sound values can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily values can be determined representative of an average daily value for the patient, either corresponding to a waking time or a 24-hour period, etc. Respiration values can include, among other things, a mean or median respiration rate, binned values of rates, and a representative value of specific rate bins, etc. Heart rate values can include an average nighttime heart rate, a minimum nighttime heart rate, etc.

The activity information can include an activity measurement of the patient, such as detected using an accelerometer, a posture sensor, a step counter, or one or more other activity sensors associated with an ambulatory medical device. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical physiologic information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g. oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

The present inventors have recognized, among other things, systems and methods to determine a health index of a patient using one or more pieces of chemical information, such as potassium or creatinine information of the patient, providing a more accurate determination of the health index or a health index value, such as in contrast to using electrical or mechanical physiologic information alone, or other non-chemical physiologic information. More accurate determinations of patient health can result in more efficient use of device resources that are controlled or dependent on such determinations, including reducing power consumption or communication or processing resources of devices and sensors, such as by increasing a specificity or sensitivity of determinations of the health index, improving control of device resources, increasing a sensitivity or specificity of alert state determinations, reducing false positive alert state determinations and device transitions or adjustments associated therewith, reducing storage or transmission of physiologic information or device transitions associated with false positive alert state determinations, etc.

In an example, the health index can be determined as a function of chemical information of the patient. For example, a composite health index can be determined as a function (e.g., a weighted function) of two or more chemical features (e.g., without electrical or mechanical features), or separately one or more physiologic features (e.g., electrical or mechanical features, etc.) and one or more chemical features.

A chemical feature, such as a value of a chemical feature or parameter, can be determined using one or more pieces of chemical information, such as sensed by or received from one or more chemical sensors. The chemical feature may include the chemical information in a raw form (e.g., a value of a sensor output, such as an electrical signal, a chemical value in base unit, such as concentrations, etc.). In other examples, the chemical feature may be determined by processing the received chemical information (e.g., converting a sensor output to a chemical value, adjusting the chemical value based on one or more factors, such as adjusting to account for a temperature, pressure, other chemical concentration, etc.), etc. For example, the chemical feature can be determined based on one or more of an amount of deviation, a direction of deviation, or a rate of change of the chemical information from one or more thresholds or baselines.

In an example, the chemical feature can be determined using a comparison of chemical information (e.g., a value determined from received chemical information) to one or more thresholds or baselines. For example, the chemical information may be compared to one or more chemical feature thresholds, such as a threshold corresponding to one or more of a relative high value or a relative low value, providing an indication of a relative high or low value of the chemical information with respect to the one or more chemical feature thresholds. The one or more threshold values can be determined, in certain examples, as a percentage change from a patient baseline (e.g., a value that is 30 percent above a patient baseline may represent a relative high value, a value that is 30 percent below a patient baseline may represent a relative low value, etc.), a deviation from a short and/or long-term patient average (e.g. a deviation above a long-term average value greater than a specified threshold may represent a relative high value, etc.), a value above or below one or more patient-specific or population thresholds (e.g., a high threshold may be determined for a specific patient or for a specified population), or combinations or permutations thereof.

In an example, if the chemical feature is above a relative high value or below a relative low value, a health index or other features or parameters in a health index can be gated or adjusted as a function of the chemical feature to indicate a worsening patient condition, such as in contrast to the health index determined without the chemical feature. In contrast, if the chemical feature is within a normal threshold of a baseline (e.g., within 30 percent above or below the baseline, etc.) or otherwise determined as between the relative high and low values, the chemical feature can be assigned a value that has little to no effect on the health index or can be used to indicate an improving and/or stable patient condition.

In an example, the one or more thresholds or baselines can include a specified predetermined value (e.g., a fixed value, which can be determined with or without input based on the patient or patient's history, with or without clinicians' input, etc.). In an example, the baseline can include a moving baseline, which can represent a moving average of the patient over a specified period of time (e.g., a predefined length of 3 days, 7 days, 14 days, 21 days, 31 days, etc.). For example, the baseline can represent a chemical feature threshold determined as a function (e.g., an average, a mean, a moving average, other central tendency, weighted central tendency, etc.) of the chemical feature over a first time period (e.g., a predefined length of 3 days, 7 days, 14 days, 21 days, 31 days, etc.).

The chemical feature can be assigned a value that indicates a worsening patient condition when the chemical feature deviates from the baseline. The indicated level of worsening patient condition can be increased if the chemical feature is one or more of deviating from the baseline more quickly (e.g., deviating from the baseline by an amount over 10 days vs. over one day) or deviating farther from the baseline. The chemical feature can be assigned a value that takes into account one or more of statistical outcomes indicated by chemical feature values, theoretical outcomes predicted by chemical feature values, clinician programmed values, etc.

In an example, a chemical feature can be determined in the shape of a statistical function, such as the logistic function (e.g., sigmoid (e.g., starts at 0 and then trends towards 1), reverse sigmoid (e.g., starts at 1 and then trends towards zero), translated sigmoid (e.g., slid along the x-axis), etc.). For example, a chemical feature can indicate a risk that generally increases as the chemical feature increases, and this can be represented with a sigmoid or other statistical function tailored to the chemical feature (e.g., the shape of the chemical feature is configured so that feature values align with relative risk to the patient based on the chemical feature, such as by tailoring (e.g., expanding in the horizontal direction, expanding in the vertical direction, sliding vertically, sliding horizontally, etc.) a statistical function to match the relative risk).

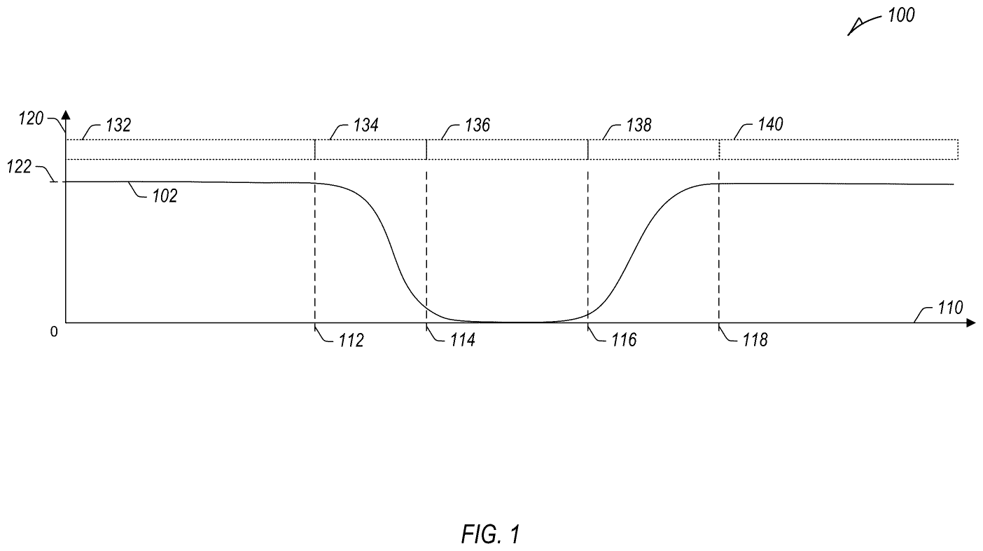

In an example, a chemical feature can have a normal value, and values above and below normal can indicate a worsening patient condition or adverse outcome. For example, potassium information can have a normal range, with deviations from the normal range above or below indicating an increasing risk of adverse outcomes. For chemical features with these and similar properties, it can be beneficial to sum statistical functions to represent the chemical feature value. For example, a sigmoid and a reverse sigmoid can be summed to generate a chemical feature to feature value mapping, such as illustrated in FIG. 1.

For example, a gated health index can be determined as a function of chemical information, providing a better indication of certain unstable, worsening, or adverse outcomes.

$$Gated Health Index = 100 * ( f ( X ) + \frac{1 - f ( X )}{100} * H ) \qquad ( 1 )$$

In function (1), ƒ(X) can be the chemical feature value (e.g., determined as a function of the chemical information X), and H can be a health index value (e.g., a health index determined without ƒ(X), a health index determined using electrical or mechanical physiologic information, a health index determined without chemical information, a health index determined using ƒ(X), the HeartLogic™ index, etc.). In an example, ƒ(X) can be a feature determined using one or more of potassium information (e.g., shown in FIG. 1) or creatinine information, and H can be the HeartLogic™ index. When the chemical feature value is neutral (e.g., 0), such as when the chemical information is normal, the gated health index can be equivalent, or nearly so, to H. As the chemical feature grows, the chemical feature determines a larger and larger portion of the health index, eventually reaching a point where the chemical feature dominates the gated health index independent of H.

In an example, the received chemical information can be used to adjust the function used to determine the health index. For example, a determined chemical feature (e.g., a chemical feature that is used in the health index, a chemical feature that is not used in the composite health index) can be used to adjust a function used to determine the composite health index. In an example, when the chemical feature indicates a worsening patient condition, the chemical feature can result in the addition of one or more other physiologic features or chemical features to the health index function that were not in the function before the chemical feature indicated a worsening patient condition. In an example, when the chemical feature indicates a worsening patient condition, a weight of one or more features in the composite health index can be adjusted (e.g., increasing a relative weight of one or more features, decreasing a relative weight of one or more features, etc.). Similar actions may be taken when a chemical feature indicates an improving combination, as well as any combination or permutation of the action for a worsening or improving indication.

In an example, the health index may have a value (e.g., a numerical value, etc.) and the value may be compared to one or more thresholds to determine the health index alert state of the system. The assessment circuit may be configured to compare the health index to one or more health index alert thresholds (e.g., a single health index alert threshold, first and second health index alert thresholds, etc.) to determine a health index alert state of the patient. In an example, the health index may have a numerical value with higher values representing a worse health state of the patient and lower values representing a better health state of the patient. The health index alert threshold may represent a high threshold that results in a health index in-alert state being triggered if the value of the health index exceeds the health index alert threshold.

The health index alert threshold may be a fixed value, or it may be an adaptable threshold that varies based on one or more factors. In an example, the health index alert threshold may be fixed, but a value of the health index may be based in part on one or more relative factors (e.g., based on measurements from the patient over the past 30 days as opposed to being based on fixed values), which may result in the in-alert state threshold condition of the patient being relative even though the health index alert threshold is fixed.

Comparing the health index value to the health index alert threshold may determine a health index alert state of the system. This health index alert state may be based at least in part on the received chemical information if the health index includes or is determined using one or more features generated using the chemical information, or if the health index alert state is affected in one or more ways by chemical information (e.g., adjusting the weighting of features in the function, adjusting the features in the function, gating the function, adjusting the alert threshold, etc.).

In some examples, if the health index value crosses over the health index alert threshold, the system may enter a health index in-alert state. The system may continue in the health index in-alert state until the health index value crosses over a health index out-of-alert threshold. The health index in-alert threshold and the health index out-of-alert threshold may have the same value (e.g., any health index value above the shared threshold results in an in-alert state being determined and any health index value below the shared threshold results in an out-of-alert state being determined). The health index in-alert threshold and the health index out-of-alert threshold may have different values. For example, the health index out-of-alert threshold (e.g., the threshold that the health index value must cross to transition from an in-alert state to an out-of-alert state once an in-alert state is determined) may be lower than the health index in-alert threshold (e.g., the threshold that the health index value must cross to transition from an out-of-alert state to an in-alert state). This may create a hysteresis for the health index alert state (e.g., once the in-alert state is determined, the health index value must drop below the health index value that was required to enter the in-alert state by a specified margin before an out-of-alert state is determined), which may prevent rapid switching between in-alert and out-of-alert states, require meaningful improvement in patient condition before transitioning to an out-of-alert state, etc.

In an example, the received chemical information can be used to adjust the health index alert threshold. For example, a determined chemical feature (e.g., a chemical feature that is used in the health index, a chemical feature that is not used in the health index) can be used to adjust the health index alert threshold. In an example, if the chemical feature indicates a worsening condition, the health index alert threshold can be adjusted to make the health index more sensitive (e.g., make it easier to cause an in-alert condition). For example, if the chemical feature is not used in the health index, the chemical feature can still affect the health index alerts by altering the health index alert threshold.

In an example, the determined value of the composite health index can exceed the value of the composite health index alert threshold independent of any physiologic features used in the function to determine the composite health index. For example, the one or more chemical features in the health index can result in a health index value that triggers an alert even if the one or more physiologic features are not indicating a worsening patient condition. The alert threshold can be exceeded independent of the one or more physiologic features for a first range of values of the one or more chemical features in the composite health index. For example, certain combinations and permutations of chemical feature values that result in the health index alert threshold being exceeded independent of the physiologic features (e.g., a health index with a single chemical feature would have a set of values (e.g., a range) of that single feature (e.g., certain points and ranges along a line), a health index with two chemical features would have a set of values of both features (e.g., points and areas of a two-dimensional plane), a health index with three chemical features would have a set of values of all three features (e.g., points and volumes in a rectangular prism), etc.) In an example, the gating function shown in equation 1 can trigger an in-alert state by crossing the alert threshold, which can include when the chemical feature is between one and a minimum value necessary to exceed the composite health index (e.g., the first range of values). For example, if the health index alert threshold is 50, the value of ƒ(X) to exceed the health index alert threshold independent of the value of H would be 0.5. Therefore, the first range of values would be when ƒ(X) from 0.5 to 1 (e.g., the system will be in the in alert state any time ƒ(X) is between 0.5 and 1, independent of a value of any other feature).

In an example, the health index alert threshold can be adjusted for a second range of values of the one or more chemical features, which can increase a sensitivity of the health index. The second range of values can be different from the first range of values, but can have a similar structure. For example, certain combinations and permutations of chemical feature values can result in adjusting the health index alert threshold (e.g., a health index with a single chemical feature would have a set of values (e.g., a range) of that single feature (e.g., certain points and ranges along a line), a health index with two chemical features would have a set of values of both features (e.g., points and areas of a two-dimensional plane), a health index with three chemical features would have a set of values of all three features (e.g., points and volumes in a rectangular prism), etc.) The adjustment to the health index alert threshold can be different for different portions of the range (e.g., a greater increase in sensitivity corresponding to chemical feature(s) that indicate a greater risk of adverse outcome, etc.), or can be uniform across the range.

In an example, the number of features in the health index can be increased for a third range of values of the one or more chemical features. This can increase a sensitivity or specificity of the health index. The third range of values can be different from the first range of values and/or the second range of values, but can have a similar structure. For example, certain combinations and permutations of chemical feature values can result in increasing the number of features in the composite health index (e.g., a health index with a single chemical feature would have a set of values (e.g., a range) of that single feature (e.g., certain points and ranges along a line), a health index with two chemical features would have a set of values of both features (e.g., points and areas of a two-dimensional plane), a health index with three chemical features would have a set of values of all three features (e.g., points and volumes in a rectangular prism), etc.) The number of features added and/or the features added can be different for different portions of the range (e.g., adding one feature when a chemical feature indicates a moderately abnormal condition and adding two features when a chemical feature indicates a severely abnormal condition, etc.), or can be uniform across the range. In an example, one or more features can be added to the HeartLogic™ index when a chemical feature (e.g., a chemical feature corresponding to potassium information, a chemical feature corresponding to creatinine information, etc.) indicates an abnormal value. This can increase an overall sensitivity of the HeartLogic™ index, or can increase a specificity or otherwise tailor the HeartLogic™ index to detect worsening patient condition in specific cases where chemical information is abnormal (e.g., different features, feature weights, and alert thresholds can be used when one or more pieces of chemical information are abnormal, which can result in the health index providing a better indication of patient condition during times of abnormal chemical levels than if the health index is not tailored (e.g., constant or tailored only on non-chemical factors) based on the chemical information (e.g., high potassium level could result in the health index monitoring, or monitoring more closely, heart rhythm metrics)).

The health index can be used to determine an indication or alert of worsening patient status, including worsening heart failure, such as driven by volume overload (e.g., excessive fluid in the body, excessive blood volume, excessive ratio of plasma in the blood, etc.), etc. Chemical values can be indicative of a patient condition.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

With respect to potassium information, a normal range (e.g., a medically acceptable range, such as determined based on medical data, a healthy range, etc.) can be defined as “normokalemia,” and can include a range of values from 3.6 to 5.2 mmol/L (e.g., millimoles per liter). Potassium levels above and/or below normokalaemia can lead to adverse outcomes. For example, a low range below normokalemia can be defined as “hypokalemia,” and can include values below 3.6 mmol/L. Moderate hypokalemia may include a range of values from 2.5 to 3.6 mmol/L. Severe hypokalemia, which can require immediate medical attention, may include values below 2.5 mmol/L. A high range above normokalemia can be defined as “hyperkalemia,” and can include values above 5.2 mmol/L. Moderate hyperkalemia may include a range of values from 5.2 to 6.5 mmol/L. Severe hyperkalemia, which can require immediate medical attention, may include values above 6.5 mmol/L.

Hypokalemia can lengthen the action potential. Hypokalemia can have a causative role in one or more processes linked to heart failure progression, such as one or more of peripheral muscle dysfunction, rhabdomyolysis, impaired vasodilation, myocardial diastolic dysfunction, atherosclerosis or diuretic resistance. Hyperkalemia can increase the risk for one or more of asystole, ventricular fibrillation, or cardiac arrest. Heart failure patients can have a high prevalence of chronic kidney disease, which can increase the risk of hyperkalemia. Application of RAAS inhibitors can further increase the risk of hyperkalemia. For these and other reasons, hypokalemia and/or hyperkalemia can be indicative of a worsening patient condition and/or predictive of adverse patient outcomes, such as hospitalization or death. This can make it beneficial to include potassium information in determining a health index (e.g., include potassium information in any respect, such as discussed above, not just limited to including a feature based on potassium in the health index).

With respect to creatinine, low kidney perfusion and/or worsened kidney function can result in increased creatinine levels (e.g., serum or interstitial creatinine levels, etc.), which can lead to fluid buildup in the lungs and/or other tissue, in certain examples worsening heart failure symptoms and/or increasing the risk of adverse outcomes (e.g., hospitalization, readmission, mortality, etc.). It may be desirable to monitor a creatinine level to determine if kidney function is affecting heart failure status. For example, increased creatinine levels (e.g., serum or interstitial creatinine levels, etc.) over time, such as a long-term trend, may indicate that renal dysfunction is contributing to volume overload, which can affect the risk of adverse patient outcomes. For these and other reasons, it can be beneficial to include creatinine information in determining a health index (e.g., include creatinine information in any respect, such as discussed above, not just limited to including a feature based on creatinine in the health index).

In certain examples, a health index alert state (e.g., a health index in-alert state, a health index out-of-alert state, etc.) may be determined and provided to the patient, a clinician, or one or more other users or devices associated with the patient. The health index alert state may be determined using the health index, such as the HeartLogic™ index. The system may provide an output of the determined health index alert state to a user interface for display to a user or to a control circuit to control or adjust a process or function of the medical device system.

In an example, the health index may include a composite heart failure index, the health index alert threshold may be a composite heart failure alert threshold, and the received chemical information may include one or more of potassium level or creatine level.

In certain examples, the techniques described above or herein can be used in various combinations or permutations. For example, combinations or permutations of techniques described above or herein can be selected based upon patient history, clinician input, etc.

FIG. 1 illustrates an example representation 100 of a chemical feature value 102 through different ranges, from a normal feature range 136 to ranges above and below the normal feature range 134, 138, and significantly above and below the normal feature range 132, 140. The chemical feature value 102 has a specified output value on a vertical axis 120 over a specified range of input values represented on a horizontal axis 110. In the example of FIG. 1, the chemical feature value 102 can be in the form of a sum of logistic functions (e.g., a first reverse logistic function apparent on the left hand side of the graph that trends from a maximum feature value 122 to 0 summed with a second logistic function apparent on the right hand side of the graph that trends from 0 to the maximum feature value 122). In an example, the chemical feature value 102 can have a maximum feature value 122 of 1 (e.g., the maximum value of an unweighted logistic function). In an example, the maximum feature value 122 can be any value. The maximum feature value can be indicative of a negative patient condition and 0 can be indicative of a stable and/or positive patient condition.

The different ranges can be separated by different boundaries, such as threshold values. For example, the boundary between the significantly below normal feature range 132 and the below normal feature range 134 can be a significantly below normal threshold 112. The boundary between the normal feature range 134 and the normal feature range 136 can be a below normal feature threshold 114. The boundary between the normal feature range 136 and the above normal feature range 138 can be an above normal feature threshold 116. The boundary between the above normal feature range 138 and the significantly above normal feature range 140 can be a significantly above normal feature threshold 118.

In an example, the input on the horizontal axis 110 to the feature shown in FIG. 1 can be a piece of chemical information, which can be a chemical feature (e.g., a chemical value). In an example, the input can be potassium level. If the input is a potassium level, the significantly below normal feature range 132 can represent a severe hypokalemia condition (e.g., indicating a serum potassium level below the significantly below normal threshold 112, such as 2.5 mmol/l). The below normal feature range 134 can represent a moderate hypokalemia condition (e.g., indicating a serum potassium level between 2.5 and 3.6 mmol/l). The normal feature range 136 can represent a normokalemia condition (e.g., indicating a serum potassium level between the below normal feature threshold 114 and the above normal feature threshold 116, such as 3.6 and 5.2 mmol/l respectively). The above normal feature range 138 can represent a moderate hyperkalemia condition (e.g., indicating a serum potassium level between the above normal feature threshold 116 and the significantly above normal feature threshold 118, such as 5.2 and 6.5 mmol/l respectively).

FIG. 1 shows that the chemical feature value 102 in the normal feature range 136 can be near zero, and the chemical feature value 102 in the significantly below normal feature range 132 and the significantly above normal feature range 140 can be near the maximum feature value 122. The chemical feature value 102 in the below normal feature range 134 and the above normal feature range 138 can smoothly trend from the maximum feature value 122 to 0 or from 0 to the maximum feature value 122, respectively.

The chemical feature shown in FIG. 1 (for example, when based on potassium level), can be used in any respect, which can include using the chemical feature as discussed above. For example, the chemical feature shown in FIG. 1 can be used in a health index, used to gate a health index, used to adjust the features of the health index, used to adjust a health index alert threshold or other threshold, etc. Although illustrated in FIG. 1 with respect to potassium information (e.g., serum potassium, interstitial potassium, etc.) in other examples, the chemical feature can include creatinine information with respect to one or more other thresholds associated with normal, above/below normal, or significantly above/below normal values, such as illustrated herein with respect to potassium information.

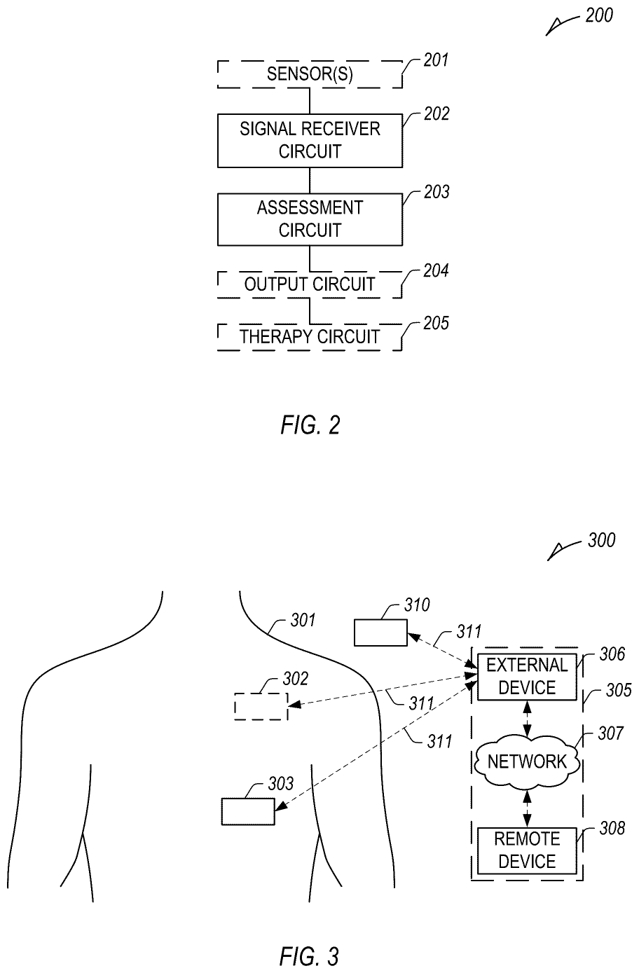

FIG. 2 illustrates an example system 200 (e.g., a medical device system). In an example, one or more aspects of the example system 200 can be a component of, or communicatively coupled to, a medical device, such as an implantable medical device (IMD), an insertable cardiac monitor, an ambulatory medical device (AMD), etc. The system 200 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 200 can include a single medical device or a plurality of medical devices implanted in a patient's body or otherwise positioned on or about the patient to monitor patient physiologic information of the patient using information from one or more sensors, such as a sensor 201. In an example, the sensor 201 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information; a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, etc.); a temperature sensor; a skin elasticity sensor, or one or more other sensors configured to receive physiologic information of the patient.

The example system 200 can include a signal receiver circuit 202 and an assessment circuit 203. The signal receiver circuit 202 can be configured to receive physiologic information of a patient (or group of patients) from the sensor 201. The assessment circuit 203 can be configured to receive information from the signal receiver circuit 202, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information.

In certain examples, the assessment circuit 203 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 200 can include an output circuit 204 configured to provide an output to a user, or to cause an output to be provided to a user, such as through an output, a display, or one or more other user interface, the output including a score, a trend, an alert, or other indication. In other examples, the output circuit 204 can be configured to provide an output to another circuit, machine, or process, such as a therapy circuit 205 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects of a medical device system, such as one or more CRT parameters, drug delivery, dosage determinations or recommendations, etc. In an example, the therapy circuit 205 can include one or more of a stimulation control circuit, a cardiac stimulation circuit, a neural stimulation circuit, a dosage determination or control circuit, etc. In other examples, the therapy circuit 205 can be controlled by the assessment circuit 203, or one or more other circuits, etc. In certain examples, the assessment circuit 203 can include the output circuit 204 or can be configured to determine the output to be provided by the output circuit 204, while the output circuit 204 can provide the signals that cause the user interface to provide the output to the user based on the output determined by the assessment circuit 203.

A technological problem exists in medical devices and medical device systems that in low-power monitoring modes, ambulatory medical devices powered by one or more rechargeable or non-rechargeable batteries (e.g., including IMDs) have to make certain tradeoffs between battery life, or in the instance of implantable medical devices with non-rechargeable batteries, between device replacement periods often including surgical procedures, and sampling resolution, sampling periods, of processing, storage, and transmission of sensed physiologic information, or features or mode selection of or within the medical devices. Medical devices can include higher-power modes and lower-power modes. Physiologic information, such as indicative of a potential adverse physiologic event, can be used to transition from a low-power mode to a high-power mode. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode. However, by the time physiologic information detected in the low-power mode indicates a possible event, valuable information has been lost, unable to be recorded in the high-power mode.

The inverse is also true, in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

For example, a change in modes can enable higher resolution sampling or an increase in the sampling frequency or number or types of sensors used to sense physiologic information leading up to and including a potential event. For example, different physiologic information is often sensed using non-overlapping time periods of the same sensor, in certain examples, at different sampling frequencies and power costs. In one example, heart sounds and patient activity can be detected using non-overlapping time periods of the same, single- or multi-axis accelerometer, at different sampling frequencies and power costs. In certain examples, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

FIG. 3 illustrates an example patient management system 300 and portions of an environment in which the patient management system 300 may operate. The patient management system 300 can perform a range of activities, including remote patient monitoring and diagnosis of a disease condition. Such activities can be performed proximal to a patient 301, such as in a patient home or office, through a centralized server, such as in a hospital, clinic, or physician office, or through a remote workstation, such as a secure wireless mobile computing device.

The patient management system 300 can include one or more medical devices, an external system 305, and a communication link 311 providing for communication between the one or more ambulatory medical devices and the external system 305. The one or more medical devices can include an ambulatory medical device (AMD), such as an implantable medical device (IMD) 302, a wearable medical device 303, or one or more other implantable, leadless, subcutaneous, external, wearable, or medical devices configured to monitor, sense, or detect information from, determine physiologic information about, or provide one or more therapies to treat various conditions of the patient 301, such as one or more cardiac or non-cardiac conditions (e.g., dehydration, sleep disordered breathing, etc.).

In an example, the implantable medical device 302 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 301. In another example, the implantable medical device 302 can include a monitor implanted, for example, subcutaneously in the chest of patient 301, the implantable medical device 302 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 302 can include an assessment circuit configured to detect or determine specific physiologic information of the patient 301, or to determine one or more conditions or provide information or an alert to a user, such as the patient 301 (e.g., a patient), a clinician, or one or more other caregivers or processes, such as described herein. The implantable medical device 302 can alternatively or additionally be configured as a therapeutic device configured to treat one or more medical conditions of the patient 301. The therapy can be delivered to the patient 301 via the lead system and associated electrodes or using one or more other delivery mechanisms. The therapy can include delivery of one or more drugs to the patient 301, such as using the implantable medical device 302 or one or more of the other ambulatory medical devices, etc. In some examples, therapy can include CRT for rectifying dyssynchrony and improving cardiac function in heart failure patients. In other examples, the implantable medical device 302 can include a drug delivery system, such as a drug infusion pump to deliver drugs to the patient for managing arrhythmias or complications from arrhythmias, hypertension, hypotension, or one or more other physiologic conditions. In other examples, the implantable medical device 302 can include one or more electrodes configured to stimulate the nervous system of the patient or to provide stimulation to the muscles of the patient airway, etc.

The wearable medical device 303 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 305 can include a dedicated hardware/software system, such as a programmer, a remote server-based patient management system, or alternatively a system defined predominantly by software running on a standard personal computer. The external system 305 can manage the patient 301 through the implantable medical device 302 or one or more other ambulatory medical devices connected to the external system 305 via a communication link 311. In other examples, the implantable medical device 302 can be connected to the wearable medical device 303, or the wearable medical device 303 can be connected to the external system 305, via the communication link 311. This can include, for example, programming the implantable medical device 302 to perform one or more of acquiring physiologic data, performing at least one self-diagnostic test (such as for a device operational status), analyzing the physiologic data, or optionally delivering or adjusting a therapy for the patient 301. Additionally, the external system 305 can send information to, or receive information from, the implantable medical device 302 or the wearable medical device 303 via the communication link 311. Examples of the information can include real-time or stored physiologic data from the patient 301, diagnostic data, such as detection of patient hydration status, hospitalizations, responses to therapies delivered to the patient 301, or device operational status of the implantable medical device 302 or the wearable medical device 303 (e.g., battery status, lead impedance, etc.). The communication link 311 can be an inductive telemetry link, a capacitive telemetry link, or a radio-frequency (RF) telemetry link, or wireless telemetry based on, for example, “strong” Bluetooth or IEEE 502.11 wireless fidelity “Wi-Fi” interfacing standards. Other configurations and combinations of patient data source interfacing are possible.

The external system 305 can include an external device 306 in proximity of the one or more ambulatory medical devices, and a remote device 308 in a location relatively distant from the one or more ambulatory medical devices, in communication with the external device 306 via a communication network 307. Examples of the external device 306 can include a medical device programmer. The remote device 308 can be configured to evaluate collected patient or patient information and provide alert notifications, among other possible functions. In an example, the remote device 308 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 308 can receive data from multiple patients. The data can be collected by the one or more ambulatory medical devices, among other data acquisition sensors or devices associated with the patient 301. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 308 may additionally include one or more locally configured clients or remote clients securely connected over the communication network 307 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 308, including the server and the interconnected clients, may also execute a follow-up scheme by sending follow-up requests to the one or more ambulatory medical devices, or by sending a message or other communication to the patient 301 (e.g., the patient), clinician or authorized third party as a compliance notification.

The communication network 307 can provide wired or wireless interconnectivity. In an example, the communication network 307 can be based on the Transmission Control Protocol/Internet Protocol (TCP/IP) network communication specification, although other types or combinations of networking implementations are possible. Similarly, other network topologies and arrangements are possible.

One or more of the external device 306 or the remote device 308 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 306 or the remote device 308 can include a respective display unit for displaying the physiologic or functional signals, or alerts, alarms, emergency calls, or other forms of warnings to signal the detection of arrhythmias. In some examples, the external system 305 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 305 can be implemented using hardware, software, firmware, or combinations thereof. Portions of the one or more ambulatory medical devices or the external system 305 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The therapy device 310 can be configured to send information to or receive information from one or more of the ambulatory medical devices or the external system 305 using the communication link 311. In an example, the one or more ambulatory medical devices, the external device 306, or the remote device 308 can be configured to control one or more parameters of the therapy device 310. The external system 305 can allow for programming the one or more ambulatory medical devices and can receives information about one or more signals acquired by the one or more ambulatory medical devices, such as received via a communication link 311. The external system 305 can include a local external implantable medical device programmer. The external system 305 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), such as in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected atrial fibrillation event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, such as to increase arterial pressure, maintain cardiac output, and to disrupt or reduce the impact of the detected atrial fibrillation event.

In certain examples, physiologic information of a patient can be sensed, such as by one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected atrial fibrillation event can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

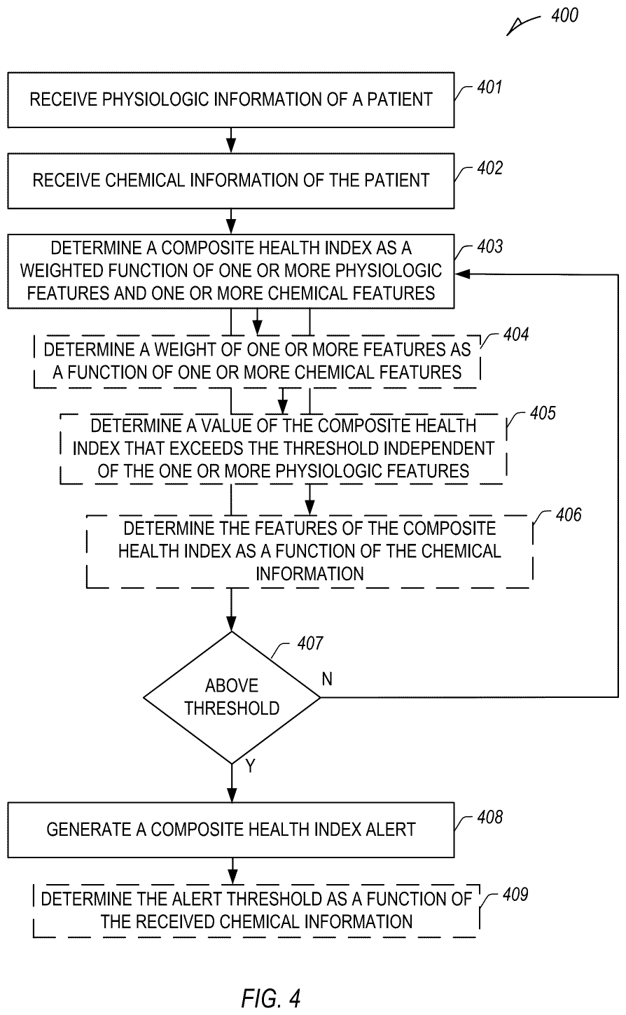

FIG. 4 illustrates an example method 400 for determining a health index using one or more pieces of chemical information. In the example of FIG. 4, the health index includes a composite health index determined using one or more pieces of physiologic information and one or more pieces of chemical information.

At step 401, physiologic information of a patient can be received, such as using a signal receiver circuit. The physiologic information of the patient can include at least one of respiration information (e.g., respiratory rate, tidal volume, RSBI, etc.), cardiac electrical information (e.g., heart rate, impedance, etc.), impedance information, cardiac acceleration information (e.g., heart sounds, etc.), mechanical acceleration information (e.g., activity information, heart sounds, etc.), mechanical position information (e.g., patient posture, sleep incline, etc.), or other physiologic information of the patient.

At step 402, chemical information of a patient can be received, such as using a signal receiver circuit. The chemical information can include information about chemicals within or other properties of a patient's blood or interstitial space, such as is otherwise discussed herein.

At step 403, the composite health index can be determined as a function of one or more features of physiologic information of the patient and one or more pieces of chemical information, such as otherwise discussed herein, for example, using an assessment circuit. The one or more physiologic features can include functions of the physiologic information received by the signal receiver circuit. For example, a feature corresponding to a breathing rate could be calculated with a high feature value corresponding to a healthy breathing rate and low feature value corresponding to an unhealthy breathing rate. A high breathing rate (e.g., 30 breaths per minute) can be assigned a value of 200 and a low breathing rate (e.g., 12 breaths per minute) can be assigned a value of 0. The function mapping between the high breathing rate and low breathing rate can be assigned one or more of linearly, logarithmically, etc.

The one or more chemical features can include functions of the chemical information received by the signal receiver circuit. A chemical feature may include a chemical value determined based on the received chemical information. The chemical feature may include the chemical information in a raw form (e.g., a sensor output, such as an electrical signal, a chemical value in base units (e.g., concentration, etc.), etc.), or the chemical feature may be determined by processing the received chemical information (e.g., converting a sensor output to a chemical value, adjusting the chemical value based on one or more factors (e.g., adjusting to account for a temperature, pressure, other chemical concentration, etc.), etc.), etc. For example, a feature based on potassium information of the patient, as discussed above.

In an example, more than one chemical feature can be used, which may include a first chemical feature and a second chemical feature. The first chemical feature and the second chemical feature can be determined, such as described above. In an example, the first chemical feature can represent a potassium level and the second chemical feature may represent a creatinine level. The health index can be determined as a function of different features or combinations of physiologic information and chemical information, such as one or more of a weighted combination (e.g., average, product, summation, etc.) of two or more features (e.g., each feature having a corresponding weight). The combination can include a linear combination or one or more non-linear or other combinations.

At step 404, a weight of one or more features of the function used to determine the composite health index can be adjusted as a function of one or more chemical features, as otherwise discussed herein. For example, a weight of the one or more chemical features in the composite health index can be determined as a function of a value of one or more of the one or more chemical features.

At step 405, a value of the composite health index that exceeds one or more thresholds discussed with respect to step 407 below can be determined independent of the one or more physiologic features, as otherwise discussed herein. For example, a value of the composite health index that exceeds a value of the composite health index alert threshold (discussed below with respect to step 7) can be determined for a first range of values of one or more of the one or more chemical features.

At step 406, the features of the composite health index can be determined as a function of the received chemical information, such as is otherwise discussed above. For example, an additional feature can be added to the function used to determine the composite health index in step 403. In an example, a feature can be removed from the function used to determine the composite health index in step 403. In an example, any combination of adding or removing features may be used (e.g., adding two features and removing one feature). For example, the method can include increasing a number of features in the composite health index for a third range of values of one or more of the one or more chemical features.

At step 407, the determined composite health index value can be compared to a composite health index alert threshold, such as using an assessment circuit. If the composite health index value is a specified side of the threshold (e.g., higher than the threshold, lower than the threshold), the method can include returning to step 403 to redetermine the composite health index. The composite health index value determined at step 403 can be determined and compared to the composite health index threshold recurrently. For example, the composite health index can be compared to the composite health index threshold at set interval, which can include 1 minute, 5 minutes, 30 minutes, 1 hour, 12 hours, or 1 day.

At step 408, if the composite health index value is the opposite side of the threshold (e.g., the opposite side of the threshold as results in returning to step 403), a composite health index in-alert state can be determined. An indication of the determined health index in-alert state may be provided to the patient, a clinician, or one or more other users associated with the patient, etc. In an example, an alert can be generated and provided of a transition or adjustment from the out-of-alert state to the in-alert state. The alert may be provided at a specified urgency based on a determined priority of the alert state (e.g., audible, visual, or haptic alarming, urgent notifications, etc.). If an in-alert state is determined, the system may remain in an in-alert state until one or more of the alert is reset or the system determines that an out-of-alert state is appropriate (e.g., the health index value falls below the health index out-of-alert threshold). During an in-alert state, the power consumption of a device may be increased, which may be due to one or more of a power required to generate and/or transmit one or more alerts, an increased monitoring interval, an increased processor load, etc.

At step 409, one or more thresholds used in step 407 to determine the composite health index alert can be determined as a function of the received chemical information, such as is discussed above. For example, the alert threshold can be adjusted to increase a sensitivity of the composite health index for a second range of values of one or more of the one or more chemical features.

In certain examples, the techniques of any one or more of steps 404-406 and 409 can be used in various combinations or permutations. In certain examples, any one or more of steps 404-406 or 409 can apply to more than one piece of chemical information.

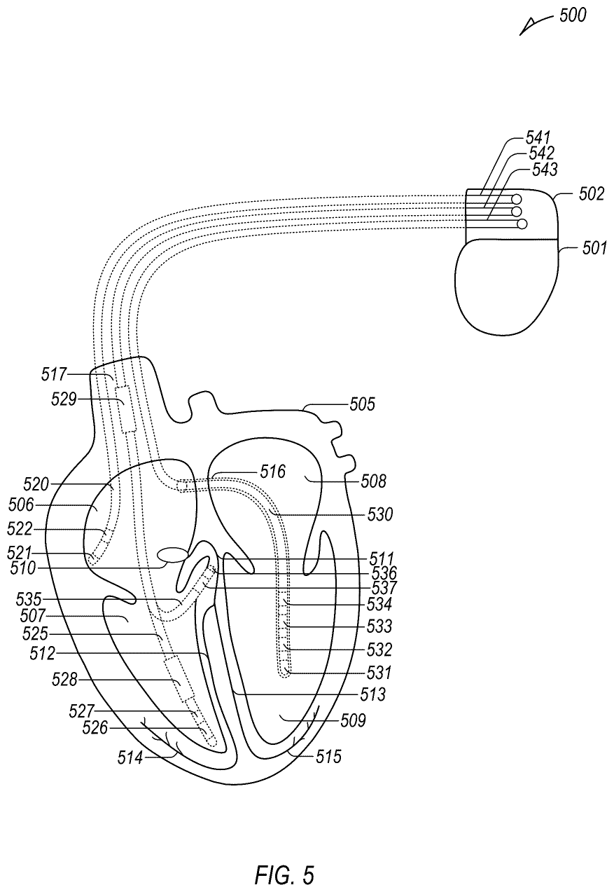

FIG. 5 illustrates an implantable medical device (IMD) 500 electrically coupled to a heart 505, such as through one or more leads coupled to the IMD 500 through one or more lead ports, such as first, second, or third lead ports 541, 542, 543 in a header 502 of the IMD 500. In an example, the IMD 500 can include an antenna, such as in the header 502, configured to enable communication with an external system and one or more electronic circuits (e.g., an assessment circuit, etc.) in a hermetically sealed housing (CAN) 501. The IMD 500 illustrates an example medical device (or a medical device system) as described herein.

The IMD 500 may include an implantable medical device (IMD), such as an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 505, such as in one or more of the atria or ventricles. Separate from, or in addition to, the one or more electrodes or other sensors of the leads, the IMD 500 can include one or more electrodes or other sensors (e.g., a pressure sensor, an accelerometer, a gyroscope, a microphone, etc.) powered by a power source in the IMD 500. The one or more electrodes or other sensors of the leads, the IMD 500, or a combination thereof, can be configured detect physiologic information from, or provide one or more therapies or stimulation to, the patient.

The IMD 500 can include one or more electronic circuits configured to sense one or more physiologic signals, such as an electrogram or a signal representing mechanical function of the heart 505. In certain examples, the CAN 501 may function as an electrode such as for sensing or pulse delivery. For example, an electrode from one or more of the leads may be used together with the CAN 501 such as for unipolar sensing of an electrogram or for delivering one or more pacing pulses. A defibrillation electrode (e.g., the first defibrillation coil electrode 528, the second defibrillation coil electrode 529, etc.) may be used together with the CAN 501 to deliver one or more cardioversion/defibrillation pulses.

In an example, the IMD 500 can sense impedance such as between electrodes located on one or more of the leads or the CAN 501. The IMD 500 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the IMD 500 can be configured to inject current between an electrode on one or more of the first, second, third, or fourth leads 520, 525, 530, 535 and the CAN 501, and to sense the resultant voltage between the same or different electrodes and the CAN 501.

The example lead configurations in FIG. 5 include first, second, and third leads 520, 525, 530 in traditional lead placements in the right atrium (RA) 506, right ventricle (RV) 507, and in a coronary vein 516 (e.g., the coronary sinus) over the left atrium (LA) 508 and left ventricle (LV) 509, respectively, and a fourth lead 535 positioned in the RV 507 near the His bundle 511, between the AV node 510 and the right and left bundle branches 512, 513 and Purkinje fibers 514, 515. Each lead can be configured to position one or more electrodes or other sensors at various locations in or near the heart 505 to detect physiologic information or provide one or more therapies or stimulation.

The first lead 520, positioned in the RA 506, includes a first tip electrode 521 located at or near the distal end of the first lead 520 and a first ring electrode 522 located near the first tip electrode 521. The second lead 525 (dashed), positioned in the RV 507, includes a second tip electrode 526 located at or near the distal end of the second lead 525 and a second ring electrode 527 located near the second tip electrode 526. The third lead 530, positioned in the coronary vein 516 over the LV 509, includes a third tip electrode 531 located at or near the distal end of the third lead 530, a third ring electrode 532 located near the third tip electrode 531, and two additional electrodes 533, 534. The fourth lead 535, positioned in the RV 507 near the His bundle 511, includes a fourth tip electrode 536 located at or near the distal end of the fourth lead 535 and a fourth ring electrode 537 located near the fourth tip electrode 536. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 525 includes a first defibrillation coil electrode 528 located near the distal end of the second lead 525 in the RV 507 and a second defibrillation coil electrode 529 located a distance from the distal end of the second lead 525, such as for placement in or near the superior vena cava (SVC) 517.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 511.

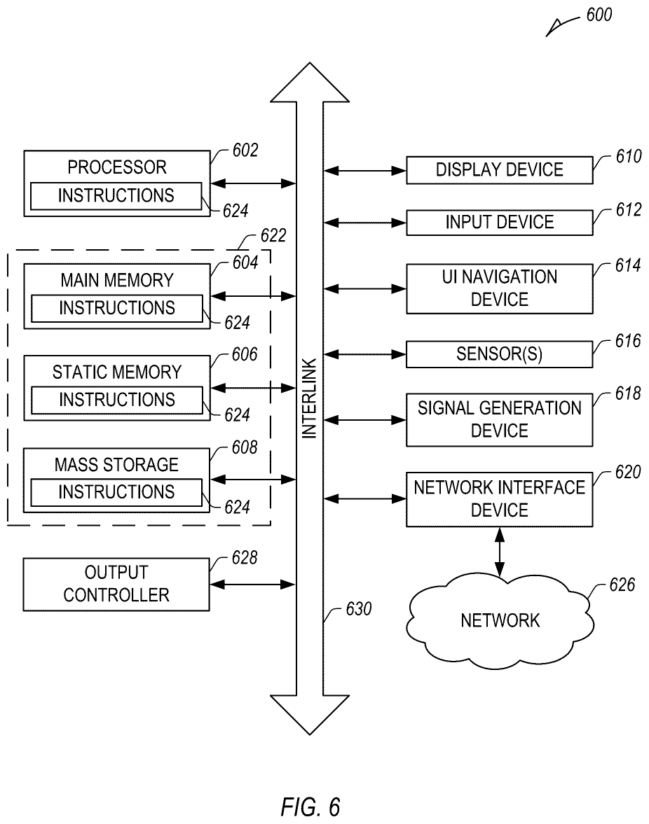

FIG. 6 illustrates a block diagram of an example machine 600 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 600. Circuitry (e.g., processing circuitry, an assessment circuit, etc.) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 600 (e.g., computer system) may include a hardware processor 602 (e.g., a central processing unit (CPU), a graphics processing unit (GPU), a hardware processor core, or any combination thereof), a main memory 604, a static memory 606 (e.g., memory or storage for firmware, microcode, a basic-input-output (BIOS), unified extensible firmware interface (UEFI), etc.), and mass storage 608 (e.g., hard drive, tape drive, flash storage, or other block devices) some or all of which may communicate with each other via an interlink 630 (e.g., bus). The machine 600 may further include a display unit 610, an input device 612 (e.g., a keyboard), and a user interface (UI) navigation device 614 (e.g., a mouse). In an example, the display unit 610, input device 612, and UI navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 602, the main memory 604, the static memory 606, or the mass storage 608 may be, or include, a machine-readable medium 622 on which is stored one or more sets of data structures or instructions 624 (e.g., software) embodying or utilized by any one or more of the techniques or functions described herein. The instructions 624 may also reside, completely or at least partially, within any of registers of the hardware processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system, comprising:
a signal receiver circuit configured to receive:
physiologic information of a patient; and
chemical information of the patient; and
an assessment circuit configured to:
determine a composite health index for the patient as a weighted function of one or more physiologic features of the received physiologic information and one or more chemical features of the received chemical information; and
generate a composite health index alert as a function of the determined composite health index and an alert threshold.

2. The medical device system of claim 1, wherein the assessment circuit is configured to determine the composite health index for the patient as a function of at least two features of the received physiologic information and one or more features of the received chemical information.

3. The medical device system of claim 1, wherein the one or more chemical features comprise one or more indications of relative high or low values of the received chemical information with respect to one or more chemical feature thresholds.

4. The medical device system of claim 3, wherein the assessment circuit is configured to:
determine the one or more chemical feature thresholds as a function of the received chemical information over a first time period; and
determine the indication of relative high or low values of the received chemical information using a comparison of a value of the received chemical information to the determined one or more chemical feature thresholds.

5. The medical device system of claim 1, wherein the assessment circuit is configured to determine a weight of the one or more chemical features as a function of a value of the one or more chemical features.

6. The medical device system of claim 5, wherein the assessment circuit is configured to determine a value of the composite health index that exceeds a value of the alert threshold for a first range of values of the one or more chemical features, independent of the one or more physiologic features.

7. The medical device system of claim 1, wherein the assessment circuit is configured to determine the alert threshold as a function of the received chemical information.

8. The medical device system of claim 7, wherein the assessment circuit is configured to determine the alert threshold to increase a sensitivity of the composite health index alert for a second range of values of the one or more chemical features.

9. The medical device system of claim 1, wherein the assessment circuit is configured to determine the one or more physiologic features or the one or more chemical features of the weighted function of the composite health index as a function of one or more values of the one or more chemical features.

10. The medical device system of claim 9, wherein the assessment circuit is configured to increase a number of features of the one or more physiologic features or the one or more chemical features of the weighted function for a third range of values of the one or more chemical features.

11. The medical device system of claim 1, wherein the composite health index includes a composite heart failure index and the composite health index alert threshold includes a composite heart failure alert threshold,
wherein at least one of the one or more chemical features or the chemical information includes at least one of potassium information or creatinine information of the patient.

12. The medical device system of claim 1, wherein the assessment circuit is configured to provide an output of the generated composite health index alert to a user interface for display to a user or to a control circuit to control or adjust a process or function of the medical device system.

13. A method, comprising:
receiving, using a signal receiver circuit:
physiologic information of a patient; and
chemical information of the patient;
determining, using an assessment circuit, a composite health index for the patient as a weighted function of one or more physiologic features of the received physiologic information and one or more chemical features of the received chemical information; and
generating, using the assessment circuit, a composite health index alert as a function of the determined composite health index and an alert threshold.

14. The method of claim 13, wherein determining the composite health index for the patient includes determining the composite health index for the patient as a function of at least two features of the received physiologic information and one or more features of the received chemical information.

15. The method of claim 13, wherein the one or more chemical features comprise one or more indications of relative high or low values of the received chemical information with respect to one or more chemical feature thresholds,
wherein the method comprises determining, using the assessment circuit:
the one or more chemical feature thresholds as a function of the received chemical information over a first time period; and
the indication of relative high or low values of the received chemical information by comparing a value of the received chemical information to the determined one or more chemical feature thresholds.

16. The method of claim 15, comprising determining, using the assessment circuit, a weight of the one or more chemical features as a function of a value of the one or more chemical features.

17. The method of claim 16, comprising determining, using the assessment circuit, a value of the composite health index that exceeds a value of the alert threshold for a first range of values of the one or more chemical features, independent of the one or more physiologic features.

18. The method of claim 13, comprising determining, using the assessment circuit, the alert threshold as a function of the received chemical information.

19. The method of claim 18, comprising determining, using the assessment circuit, the alert threshold to increase a sensitivity of the composite health index alert for a second range of values of the one or more chemical features.

20. The method of claim 13, comprising determining, using the assessment circuit, the one or more physiologic features or the one or more chemical features of the weighted function of the composite health index as a function of one or more values of the one or more chemical features.
